(19) United States
(12) Patent Application Publication
(10) Pub. No.: US 2003/0216582 A1
(43) Pub. Date: Nov. 20, 2003

(54) 2-CARBOXAMIDE-BENZIMIDAZOLES USEFUL IN THE TREATMENT AND PREVENTION OF ISCHEMIC REPERFUSION INJURY

(76) Inventors: Nicholas Nikolaides, Mason, OH (US); Donatella Chianelli, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/203,246

(22) PCT Filed: Feb. 8, 2001
(86) PCT No.: PCT/US01/04211

(51) Int. Cl. C07D 235/28; A61K 31/4178

(52) U.S. Cl. 548/309.4; 514/394

(57) ABSTRACT

The subject invention relates to compounds having the structure: (I) wherein: (a) R1 is selected from the group consisting of alkyl, aryl, alkoxy, and aryloxy, the alkyl and aryl portions of preferred R1 having from 1 to about 14 carbon atoms; (b) R3 and R4 are independently selected from the group consisting of hydrogen, halo, alkyl, alkoxy, alkyl-aryloxy, alkylthio, amino, and mono- or dialkylaminio, the alkyl portions of preferred R3 and R4 having from 1 to about 8 carbon atoms; except that R3 and R4 are not both hydrogen; (c) each R5 is independently selected from the group consisting of hydrogen, halo, cyano, alkyl, hydroxy, alkoxy, thio, alkylthio, amino, and mono- or dialkylamino, the alkyl portions of preferred R5 having from 1 to about 8 carbon atoms. The subject invention further relates to compositions comprising these compound. The subject compounds are useful for preventing or treating reperfusion injury of a variety of tissues.

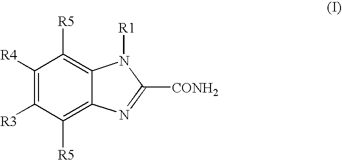

### CROSS REFERENCE TO RELATED APPLICATION

This application claims the benefit of U.S. Provisional Application Serial No. 60/181,234, 7945P, filed on Feb. 9, 2000.

### FIELD OF THE INVENTION

The subject invention relates to 2-carboxamide-benzimidazoles useful for treatment or prevention of ischemic reperfusion injury of myocardial and other tissue and other cardiovascular and inflammatory diseases and disorders.

### SUMMARY OF THE INVENTION

The subject invention includes compounds having the structure:

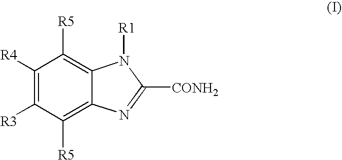

wherein:

(a) R1 is selected from the group consisting of alkyl, aryl, alkoxy, and aryloxy, the alkyl and aryl portions of preferred R1 having from 1 to about 14 carbon atoms;

(b) R3 and R4 are independently selected from the group consisting of hydrogen, halo, alkyl, alkoxy, alkyl-aryloxy, alkylthio, amino, and mono- or dialkylaminio, the alkyl portions of preferred R3 and R4 having from 1 to about 8 carbon atoms; except that R3 and R4 are not both hydrogen;

(c) each R5 is independently selected from the group consisting of hydrogen, halo, cyano, alkyl, hydroxy, alkoxy, thio, alkylthio, amino, and mono- or dialkylamino, the alkyl portions of preferred R5 having from 1 to about 8 carbon atoms; and

an optical isomer, diastereomer, or enantiomer or mixture thereof; a pharmaceutically-acceptable salt, hydrate, or biohydrolyzable ester, amide or imide thereof.

The present invention further relates to pharmaceutical compositions containing such compounds; and methods of using such compounds for treating or preventing reperfusion injuries to tissues.

### DETAILED DESCRIPTION OF THE INVENTION

There are about 1.1 million myocardial infarctions in the United States each year and 350,000 people die. Myocardial infarction (MI), or heart attack, occurs when the blood vessels supplying the heart become completely or partially occluded. Treatment with thrombolytic agents to restore blood flow (reperfusion) is first line treatment in many cases. However, the benefit of reperfusion is compromised by the acute inflammatory response associated with it, resulting in a syndrome called reperfusion injury.

Inflammation generally serves a protective role. For example, at sites of bacterial infection, bacterial endotoxins induce the production of inflammatory cytokines which recruit circulating leukocytes, including neutrophils and monocytes, to destroy the bacteria. Once the infection is cleared, the inflammation subsides. However, there are conditions where the inflammatory signal is sustained (rheumatoid arthritis) or is unnecessarily severe (ischemia-reperfusion injury).

An essential feature of inflammation is the migration of neutrophils (PMNs) from blood into tissues. This migration is preceded by a cascade of events mediated by adhesion molecules. The adherence of PMNs to vascular endothelial cells requires the interaction of adhesion molecules on the surface of both cell types. These molecules belong to three distinct families: the selectins, the integrins and the immunoglobulin superfamily. Neutrophils first roll along endothelial cells, a process mediated by the selecting. At sites of inflammation, firm adherence is mediated by the interaction of β2 integrins on PMNs and ICAM-1 (intercellular adhesion molecule-1) expressed on the endothelial cells. Finally, transendothelial migration of PMNs into tissues leads to tissue damage. Compounds able to block the adhesion of neutrophils to endothelium would be useful in the treatment of a variety of conditions involving ischemia-reperfusion injury including, but not restricted to, myocardial infarction, coronary artery bypass grafting, angioplasty, angina, stroke, peripheral vascular disease, inflammatory bowel disease, ulcerative colitis, burns, frostbite, adult respiratory distress syndrome, asthma, tissue and organ transplants, general surgery, replantation, acute renal failure, rheumatoid arthritis, psoriasis, hepatitis, pancreatitis, sunburn, radiation, ulcer, and shock. (For recent reviews see: C. Cornejo, J. Harlan, R. Winn, in Adhesion Molecules in Health & Disease, L. Paul and T. Issekutz, Eds., Marcel Dekker, 1997, Chapter 18; J. Prince, C. Ballantyne, Emerging Therapeutic Targets, 1999, 263-277.)

### Glossary Of Terms

Unless otherwise specified, the following terms have the indicated meanings when used in this application.

The term “alkyl” means a hydrocarbon chain which is linear, branched or cyclic, saturated or unsaturated (but not aromatic), substituted or unsubstituted. The term may be used alone or as part of another word where it may be shortened to “alk” (e.g., in alkoxy, alkylamino). Preferred linear alkyl have from 1 to about 20 carbon atoms, more preferably from 1 to about 8 carbon atoms, more preferably still from 1 to about 4 carbon atoms; most preferred are methyl or ethyl. Preferred cyclic and branched alkyl have from 3 to about 20 carbon atoms, more preferably from 3 to about 10 carbon atoms, more preferably still from 3 to about 6 carbon atoms. Preferred cyclic alkyl have one hydrocarbon ring, but may have two, three, or more, fused or spirocyclic hydrocarbon rings. Alkyl may be unsaturated only with one or more double bonds (“alkenyl”) (no triple bonds), preferably with one, two, or three double bonds, more preferably with one double bond. Alkyl may be unsaturated with one or more triple bonds (“alkynyl”), preferably with one triple bond. More preferred alkyl are saturated (“alkanyl”). The term “alkylene” means an alkyl which is attached to 2 or more moieties. Preferred substituents of alkyl include alkyl, aryl, halo, hydroxy, alkoxy, aryloxy, amino, alkylamino, arylamino, thio, alkylthio, arylthio, acyl, alkylacyl, arylacyl, carboxy, alkylester, arylester, amino, alkylamino, arylamino, sulfonyl, alkylsulfonyl, arylsulfonyl, nitro, cyano, heterocycle. Preferred alkyl are unsubstituted.

The term “aryl” means an aromatic hydrocarbon ring which is substituted or unsubstituted. The term may be used alone or as part of another word (e.g., in aryloxy, arylamino). Preferred aryl have from 6 to about 14 carbon atoms in the aromatic ring(s), and a total of from about 6 to about 20, preferably to about 12, carbon atoms. Preferred aryl is phenyl or naphthyl; most preferred is phenyl. The term “arylene” means an aryl which is attached to two or more other moieties. Preferred substituents of aryl include alkyl, aryl, halo, hydroxy, alkoxy, aryloxy, amino, alkylamino, arylamino, thio, alkylthio, arylthio, acyl, alkylacyl, arylacyl, carboxy, alkylester, arylester, amino, alkylamino, arylamino, sulfonyl, alkylsulfonyl, arylsulfonyl, nitro, cyano, heterocycle.

The term “heteroatom” means a nitrogen, oxygen, or sulfur atom.

The term “heterocycle” or “heterocyclyl” means a cyclic alkyl or aryl with one or more heteroatoms substituted for carbon atoms in the ring(s), preferably 1, 2 or 3 heteroatoms in the ring(s). Preferred heterocycle substituents are the same as for alkyl. The term “heteroaryl” refers to the subset of heterocycele which comprise an aromatic ring. Preferred heteroaryl have from 5 to about 14, more preferably to about 10, more preferably still 5 or 6, carbon plus heteroatoms in the ring(s), and a total of from 5 to about 20, more preferably to about 12, carbon plus heteroatoms.

The term “safe and effective amount” means an amount of a pharmacologically active compound sufficient to significantly induce a positive modification in the condition to be treated, but low enough to avoid serious side effects (at a reasonable benefit/risk ratio), within the scope of sound medical judgment. A safe and effective amount of a compound will vary with the particular condition being treated, the size and age and physical condition of the patient, the severity of condition, the duration of the treatment, the nature of concurrent therapy, the particular pharmaceutically-acceptable carrier utilized, and like factors within the knowledge and expertise of the attending physician.

The term “pharmaceutically-acceptable carrier” or “pharmaceutically-acceptable excipients” means one or more compatible solid or liquid excipients which are suitable for administration to a human or lower animal. The term “compatible” means that the excipients are capable of being commingled with the pharmacologically active compound or compounds, and with each other, in a manner such that there is no interaction which substantially reduces the pharmaceutical efficacy of the composition under ordinary use situations. The excipients do not have substantial pharmacological activity themselves, but may function, for example, as diluents, lubricants, disintigration enhancers, dissolution enhancers, encapsulating materials, preservatives, colorants, flavorants, and the like. Pbarmaceutical-acceptable excipients must be of sufficiently high purity and sufficiently low toxicity to render them suitable for administration to the human or lower animal being treated.

The term “unit dosage form” means a composition comprising an amount of a pharmacologically active compound that is suitable for administration to a human or a lower animal subject in a single dose, according to good medical practice.

A “biohydrolyzable ester” is an ester of a carboxylic acid-containing 2-carboxamide benzimidazoles of the present invention that does not interfere with the activity of the 2-carboxamide benzimidazoles or that is readily converted by an animal to yield an active 2-carboxamide benzimidazole of this invention. Such esters include lower alkyl esters, lower acyloxy-alkyl esters (such as acetoxymethyl, acetoxyethyl, aminocarbonyloxymethyl, pivaloyloxymethyl and pivaloyloxyethyl esters), lactonyl esters (such as phthalidyl and thiophthalidyl esters), lower alkoxyacyloxyalkyl esters (such as methoxycarbonyloxymethyl, ethoxycarbonyloxyethyl and isopropoxycarbonyloxyethyl esters), alkoxyalkyl esters, choline esters and alkyl acylamino alkyl esters (such as acetamidomethyl esters).

### The Compounds

The subject invention involves 2-carboxamide benzimidazoles compounds having the structure:

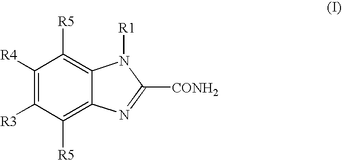

In structure (I), R1 is selected from alkyl, aryl, alkoxy, and aryloxy. The alkyl and aryl portions of preferred R1 moieties have from 1 to about 14 carbon atoms.

More preferred R1 is selected from unsubstituted or substituted alkyl having from 1 to about 12 carbons atoms, and unsubstituted or substituted phenyl or naphthyl.

More preferred alkyl R1 include unsubstituted or substituted linear alkyl having from about 2 to about 8 carbon atoms, more preferably still from about 3 to about 6 carbon atoms; still more preferred is n-propyl or n-butyl or n-pentyl. More preferred R1 include branched alkyl having from about 3 to about 8 carbon atoms, more preferably still from about 3 to about 6 carbon atoms; still more preferred is isobutyl or isopentyl. More preferred R1 include cyclic alkyl having from 3 to about 8 carbon atoms, more preferably still from 3 to about 6 carbon atoms. Preferred substituents for such linear, branched or cyclic alkyl include halo, hydroxy, alkoxy, amino, mono- and dialkylamino, thio, alkylthio, aryl (especially phenyl), and heterocycle; more preferred is such alkyl being unsubstituted. Preferred R1 which are linear, branched or cyclic alkyl are saturated or unsaturated with one or more double bonds; more preferred are saturated.

More preferred aryl R1 include unsubstituted or substituted phenyl. Preferred substituents for such phenyl include halo, hydroxy, alkoxy, amino, mono- and dialkylamino; also preferred is for such phenyl being unsubstituted.

More preferred aralkyl R1 include unsubstituted or substituted benzyl. Preferred substituents for such benzyl include halo, hydroxy, alkoxy, amino, mono- and dialkylamino; also preferred is for such benzyl being unsubstituted.

In structure (I), R3 and R4 are independently selected from the group consisting of hydrogen, halo, alkyl, alkoxy, alkyl-aryloxy, alkylthio, amino and mono- or dialkylamino, except that R3 and R4 are not both hydrogen. The alkyl portions of preferred R3 and R4 moieties have from 1 to about 8 carbon atoms. Preferably, R3 and R4 are independently selected from the group consisting of hydrogen, halo, alkyl, alkoxy, alkylthio, and mono- or dialkylamino except that R3 and R4 are not both hydrogen.

Preferred R3 and R4 include hydrogen, alkoxy having from 1 to about 6, preferably to about 3, carbon atoms; alkylthio having from 1 to about 6, preferably to about 3, carbon atoms; monoalkylamino or dialkylamino each alkyl having from 1 to about 6, preferably to about 3, carbon atoms; and alkyl having from 1 to about 6, preferably to about 3, carbon atoms. Preferred substitutents on the alkyl of such moieties include halo, hydroxy, alkoxy, amino, mono- or dialkylamino, thio, alkythio; more preferred is for such R3 and R4 moieties to be unsubstituted. More preferred still is for at least one of R3 and R4, to be ethoxy or especially methoxy. Preferably one of R3 and R4 is hydrogen; more preferably R3 is hydrogen.

In structure (I), R5 denotes moieties at positions 4 and 7 of the benzimidazole rings. Each R5 is independently selected from hydrogen, halo, cyano, alkyl, hydroxy, alkoxy, thio, alkylthio, amino, and mono- or dialkylamino. The alkyl portions of preferred R5 moieties have from 1 to about 8 carbon atoms.

Preferred R5 include hydrogen, halo, alkyl having from 1 to about 6, preferably to about 3, carbon atoms, alkoxy having from 1 to about 6, preferably about 3, carbon atoms, monoalkyl- or dialkylamino each alkyl having from 1 to about 6, preferably to about 3, carbon atoms, and alkylthio having from 1 to about 6, preferably to about 3, carbon atoms. Preferred substituents on the alkyl of such R5 moieties include alkoxy, amino, and alkyl; more preferred is for the alkyl of such moieties to be unsubstituted.

More preferred is for each R5 to be independently selected from hydrogen, halo, and unsubstituted alkyl having from 1 to about 3 carbon atoms. More preferred is for no more than one R5 being other than hydrogen. Most preferred is both R5 being hydrogen.

Preferred compounds of the subject invention include the following examples having structure (I) and the indicated substituents:

| Example | R1 | R3 | R4 | R5 |
|---|---|---|---|---|
| 1 | cyclopropyl | H | methoxy | both H |
| 2 | cyclobutyl | H | methoxy | both H |
| 3 | cyclopentyl | H | methoxy | both H |
| 4 | cyclohexyl | H | methoxy | both H |
| 5 | isopropyl | H | methoxy | both H |
| 6 | isobutyl | H | methoxy | both H |
| 7 | isopentyl | H | methoxy | both H |
| 8 | n-propyl | H | methoxy | both H |
| 9 | n-butyl | H | methoxy | both H |
| 10 | methoxyethyl | H | methoxy | both H |
| 11 | isobytyl | H | amino | both H |
| 12 | isobytyl | methoxy | H | both H |
| 13 | isobutyl | H | 4-methoxy- | Both H |
| | | | benzylamino | |
| 14 | isobutyl | H | 2-amino-1-oxo | Both H |
| | | | 3-phenyl- | |
| | | | propylamino | |

Subject invention compounds include all active optical isomers, diastereomers and enantiomers, and mixtures thereof, of the above compounds. Subject invention compounds include pharmaceutically-acceptable salts, hydrates, and biohydrolizable esters, amides, and imides of such compounds.

### Synthesis Of Compounds

The following provides general schemes for making subject invention compounds, and specific methods for synthesizing preferred subject invention compounds. Unless otherwise, stated, all commercially available reagents are used without further purification. Reactions are generally run under an inert atmosphere (argon or nitrogen). Brine refers to saturated aqueous sodium chloride. Residual solvent is removed under vacuum (ca. 0.03 mm Hg) at room temperature (rt).

Structures of the compounds synthesized are confirmed using the following analytical tools. Proton NMR spectra are taken on a GE QE-300 (300 MHz) spectrometer, a Bruker AC-300 (300 MHz) quad-nuclei probe system or on a Varian Unityplus (300 MHz). All chemical shifts are reported in δ scale as parts per million (ppm) downfiled from (CH₃)₄Si. Spectra taken in CDCl₃ are referenced either to (CH₃)₄Si or to a residual CHCl₃ (7.24 ppm). Spectra taken in D₂O are referenced to HOD (4.80 ppm), those in CO₃)₂CO are referenced to residual (CH₃)₂CO (2.04 ppm), those in CD₃OD are referenced to residual CH₃OH (3.30 ppm), and those in (CD₃)₂SO are referenced to residual (CH₃)₂SO (2.49 ppm). Carbon-13 spectra are taken on a GE QE-300 (75 MHz) spectrometer or a Bruker AC-300 (75 MHz) quad-nuclei probe system. Spectra taken in CDCl₃ are referenced to solvent (78 ppm), those in CD₃OD are referenced to solvent (49 ppm), those in (CD₃)₂SO are referenced to solvent (39.7 ppm), and those in (CD₃)₂CO are referenced to solvent (206.5, 29.8 ppm). Mass spectra are determined on a Fision's Trio 2000 equipped with a robotic probe or a Fisons Platform 11 Mass Spectrometer. Chemical ionization spectra are obtained using methane and/or ammonia as a reagent gas. ESI compound introduction is via Hewlett Packard 1050 HPLC autosampler using methanol, 0.2% formnic acid, and 0.2 mM ammonium acetate as the eluting solvent. Thin layer chromatography is performed on silica gel 60-F254 precoated plates. Flash chromatography is performed using silica get 60 (Merck, 230-400 mesh). Melting points are obtained with an Electrothermal 1A9200 or a MelTemp II capillary melting point apparatus and are uncorrected.

Scheme I is a general scheme useful for synthesizing many subject invention compounds:

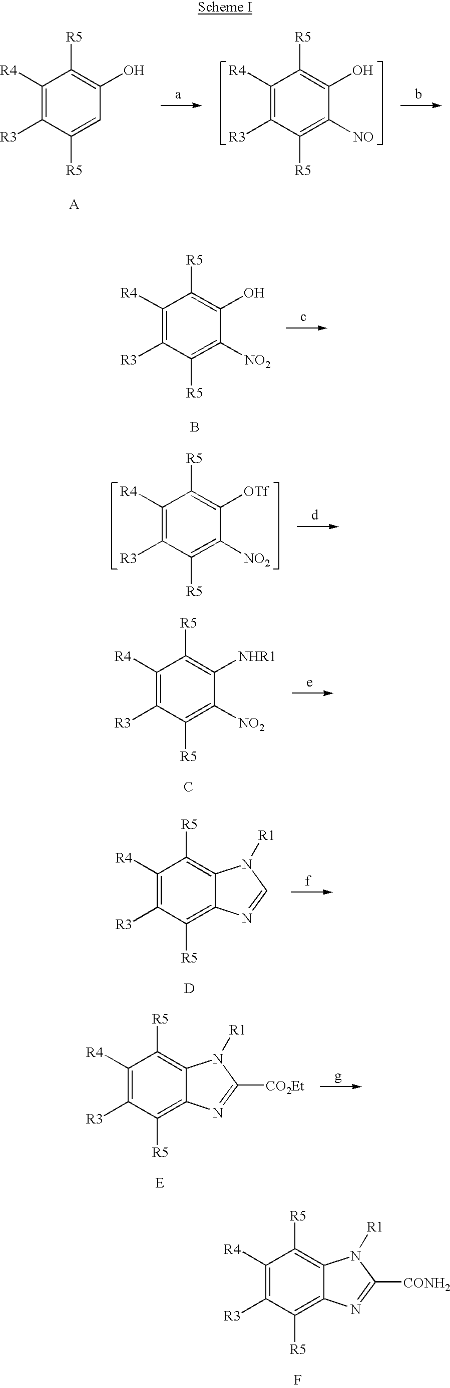

a: NaNO₂, propionic acid; b: HNO₃; c: Tf₂O/Et₃N, toluene; d: R1-NH₂; e: HCOOH, 10% Pd/C; f: n-BuLi, ECF; g: NH₃, MeOH, KCN.

5-Methoxy-2-nitrophenol (B from Scheme I with R4=methoxy and R3 and both R5=H): To a 2 liter round bottom flask, fit with a mechanical stirrer and an additional funnel, add propionic acid (300 mL) and 3-methoxyphenol (37.2 g, 0.3 mol). The resulting mixture is cooled to 0° C. and a solution of sodium nitrite (21 g, 0.304 mol) in water (50 ml) is slowly added. After stirring for 1 hr at 0° C., fuming nitric acid (40 mL) is slowly added. The resulting slurry is stirred at 0° C. for 1 hour and then warmed to room temperature over 2 hours. Water (250 mL) is added dropwise at room temperature, and the resulting solid is filtered and washed with 300 mL 50% aqueous propionic acid to provide, after drying, 5-methoxy-2-nitrophenol as a solid.

N-Alkyl-5-methoxy-2-nitroaniline (C from Scheme I with R1=alkyl, R4=methoxy, and R3 and both R5=H): To a one-liter round bottom flask, toluene (300 mL), 5-methoxy-2-nitrophenol (5.0 g., 0.03 mol), and triethylamine (6.68 g, 0.066 mol) are added. The resulting solution is then cooled to 0° C. and triflic anhydride (Tf₂O) is slowly added via syringe (9.3 g, 0.033 mol). The reaction mixture is stirred at 0° C. for 5 minutes; the amine (R1-NH₂) (0.12 mol) is added and the reaction mixture is heated to reflux for 5.5 hrs. After cooling to room temperature, the reaction content is filtered through a plug of silica gel (eluted with 90:10 hexane:ethyl acetate) and concentrated via rotary evaporation to provide crude N-alkyl-5-methoxy-2-nitroaniline. The material is used as is in the next synthetic step.

N1-Alkyl-6-methoxybenzimidazole (D from Scheme I with R1=alkyl, R4=methoxy, and R3 and both R5=H): To a 250 mL round bottom flask is added 88% formic acid (50 mL) and N-alkyl-5-methoxy-2-nitroaniline (0.02 mol). To this homogeneous mixture is added an ethyl acetate slurry of 10% Pd-C (600 mg). The resulting heterogeneous reaction mixture is heated to 100° C. for one hour, cooled to room temperature, and filtered through Celite (elute with water). The filtrate is then made basic with the addition of 28% NH₄OH and then washed with ethyl acetate (3×100 mL). The combined organics are dried (MgSO₄), filtered, and concentrated via rotary evaporation to give a residue. The residue is chromatographed (SiO₂, 50:50 hexane:ethyl acetate) to provide N1-alkyl-6-methoxybenzimidazole.

Ethyl 1-alkyl-6-methoxy-1H-benzimidazole-2-carboxylate (E from Scheme I with R1=alkyl, R4=methoxy, and R3 and both R5=H): To a 250 mL round bottom flask under argon, N1-alkyl-6-methoxybenzimidazole (2.9 g, 0.014 mol) and THF (100 mL) is added. This is cooled to −78° C. and n-butyl lithium (12.3 mL, 0.019 mol, in hexanes) is added dropwise, resulting in a yellow mixture. Stirring is continued at −78° C. for 30 minutes, then ethyl chloroformate (ECF) is added neat (2.13 g, 0.019 mol). The reaction mixture is warmed to room temperature and stirred for 30 minutes. Water (100 mL) is added and the resulting mixture extracted with ethyl acetate (3×100 mL). The combined organics are dried (MgSO₄), filtered, and concentrated to give a yellow residue. This residue is then chromatographed (flash, 70:30 hexane:ethyl acetate) to give ethyl 1-alkyl-6-methoxy-1H-benzimidazole-2-carboxylate.

N1-Alkyl-6-methoxybenzimidazole-2-carboxamide (F from Scheme I with R1=alkyl, R4—methoxy, and R3 and both R5=H): To an ACE GLASS pressure tube with stir bar, ethyl 1-alkyl-6-methoxy-1H-benzimidazole-2-carboxylate (1.8 mmol), methanolic ammonia (10 mL, 2.0 M), and sodium or potassium cyanide (8.8 mg, 0. 1 8 mmol) are added. The reaction vessel is then sealed, heated to 80° C. for 3 hours, and then cooled to room temperature. The resulting reaction mixture is concentrated via rotary evaporation, and the residue is chromatographed (flash, 60:40 hexane:ethyl aceate) to give N1-alkyl-6-methoxybenzimidazole-2-carboxamide.

Scheme II is another general scheme useful for synthesizing many subject invention compounds.

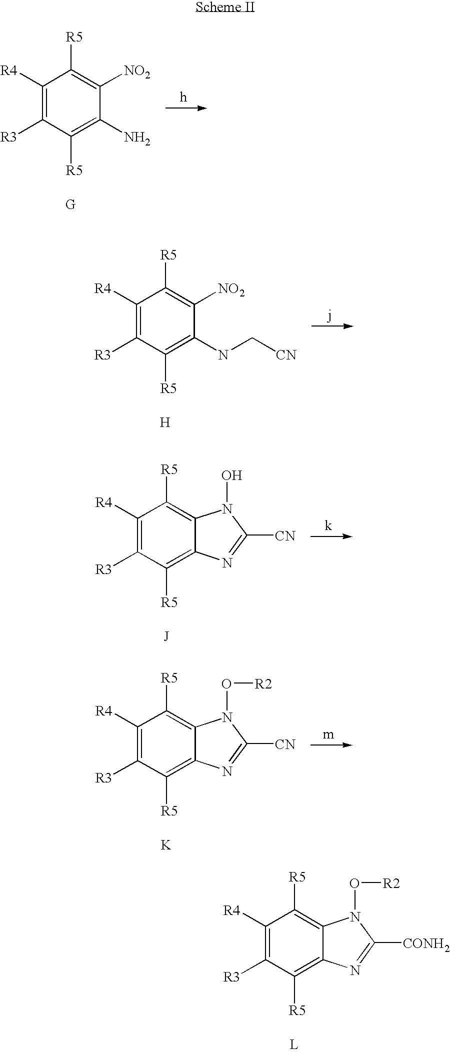

h: (CH₂O)ₙ, KCN, HOAc, ZnCl₂; j: EtOH, KOH; k: R2-OH, DEAD, Ph₃P, THF; m:KOH, t-BuOH.

N-(Cyanomethyl)-4-methoxy-2-nitroaniline (H from Scheme II with R4=methoxy and R3 and both R5=H): To a 3-neck 1L round bottom flask fitted with a mechanical stirrer, reflux condenser, and an addition funnel is added 4-methoxy-2-nitroaniline (8.4 g, 0.05 mol), paraformaldehyde (4.5 g, 0.15 mol), potassium cyanide (9.75 g, 0.15 mol), and zinc chloride (25 g, 0.18 mol). To this vigorously stirred mixture is slowly added a mixture of H₂SO₄ (4 drops) and glacial acetic acid (250 mL). The resulting reaction mixture is heated to 50° C. for 8 hours then cooled to room temperature. The reaction contents are poured over ice/water, and the solid formed is collected via suction filtration, washed with water, dried, and recrystallized (abs. EtOH) to give N-(cyanomethyl)-4-methoxy-2-nitroaniline as needles.

2-Cyano-6-methoxybenzimidazole-N1-oxide (J from Scheme II with R4=methoxy and R3 and both R5=H): To a 500 mL round bottom flask is added N-(cyanomethyl)-4-methoxy-2-nitroaniline (2.5 g, 0.012 mol) and 95% ethanol (130 mL). The reaction mixture is heated to 60° C., solid potassium carbonate (1.77 g, 0.013 mol) is added and the resulting mixture is then heated to reflux for 4.5 hours, cooled to room temperature and concentrated in vacuo. The residue is dissolved in water and then acidified (conc. HCl) to form a white solid that is collected via filtration, dried, and recrystallized (DMF) to give 2-cyano-6-methoxybenzimidazole-N-oxide as a solid.

2-Cyano-1-isopropoxy-6-methoxybenzimidazole (K from Scheme II with R1=isopropyl, R4=methoxy, and R3 and both R5=H): To a 100 mL round bottom flask is added 2-cyano-6-methoxybenzimidazole-N-oxide (457 mg, 2.41 mmol), tetrahydorfuran (THF) (50 mL), isopropyl alcohol (145 mg, 2.41 mmol), triphenylphosphine (Ph₃P) (632 mg, 2.41 mmol), and finally diethylazodicarboxylate (DEAD) (444 mg, 2.55 mmol). The resulting mixture is stirred at room temperature for 18 hours, then concentrated in vacuo. The residue is chromatographed (80:20 hexane:ethyl acetate) to give 2-cyano-1-isopropoxy-6-methoxybenzimidazole as a solid.

1-Isopropoxy-6-methoxybenzimidazole-2-carboxamide (L from Scheme II with R1=isopropyl, R4=methoxy, and R3 and both R5=H): To a 25 mL round bottom flask is added 2-cyano-1-isopropoxy-6-methoxybenzimidazole (100 mg, 0.43 mmol), t-butyl alcohol (5 mL), and potassium hydroxide (200 mg, 3.57 mmol). This mixture is refluxed for 1 hour, cooled to room temperature, poured into brine (50 mL), and extracted with ethyl acetate (3×50 mL). The combined organic fractions are dried (MgSO₄), filtered, and concentrated in vacuo to give a yellow oil. This oil is chromatographed (70:30 hexane:ethyl acetate) to provide 1-isopropoxy-6-methoxybenzimidazole-2-carboxamide as a solid.

Scheme III is another general scheme useful for synthesizing many subject invention compounds:

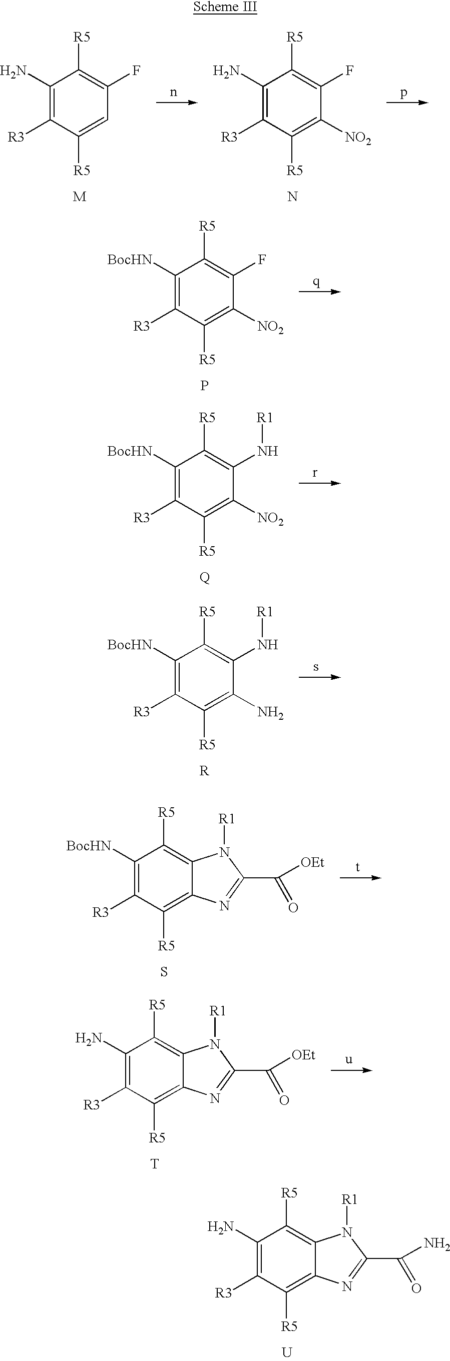

n: PhCHO, HNO₃, H₂SO₄; p: (Boc)₂CO, CH₂Cl₂; q: R1-NH₂, CH₃CN; r: 10% Pd/C, EtOH; s: (CHO-COOEt)ₙ/toluene, I₂/EtOH; t: CF₃COOH, CH₂Cl₂; u: NH₄OH, THF.

3-Fluoro-4-nitroaniline (N from Scheme III with R4=amino and R3 and both R5=H): 3-Fluoroaniline (5 g; 1 equivalent) is combined with benzaldehyde (5 g; 1.05 equivalent). The mixture is stirred at 25° C. for ½ h while achieving a gentle reflux. The mixture is dissolved in concentrated sulfuric acid (20 ml) and cooled to <5° C. with an ice bath. A mixture of concentrated nitric acid (3 ml) and concentrated sulfuric acid (10 ml) is added slowly and the mixture is stirred at 25° C. for 1½ h. The reaction is quenched by pouring onto water (200 ml); this is then washed with ethyl acetate. The combined ethyl acetate layers are dried over magnesium sulfate and evaporated to dryness. The product is chromatographed on a silica gel column using 20% ethyl acetate-hexane as the eluant to give the title compound and a mixture of other regioisomers.

(3-Fluoro4-nitro-phenyl)-carbamic acid tert-butyl ester (P from Scheme III with R4=Boc-NH and R3 and both R5=H): Ditertbutyldicarbonate (1.4 g; 1 equivalent) is combined with 3-fluoro-4-nitroaniline (1 g; 1 equivalent) in dichloromethane (30 ml). The mixture is stirred at 25° C. for 10 min at which point dimethylaminopyridine (0.08 g; 0.1 equivalent) is added. The mixture is stirred at 25° C. for 24 h. The mixture is diluted in ethyl acetate and washed with 1 molar aqueous citric acid; the mixture is then washed twice with water. The ethyl acetate layer is dried over magnesium sulfate and evaporated to dryness. The product is chromatographed on a silica gel column using 30% ethyl acetate-hexane as the eluant to give a mixture of the title compound (and the bis Boc protected compound).

(3-Isobutylamino-4-nitro-phenyl)-carbamic acid tert-butyl ester (Q from Scheme III with R1=isobutyl, R4=Boc-NH, and R3 and both R5=H): Isobutylamine (0.29 g; 2 equivalents) is added slowly to a solution of (3-fluoro-4-nitrophenyl)-carbamic acid tert-butyl ester (0.5 g; 1 equivalent) in acetonitrile (20 ml). The mixture is stirred at 25° C. for 24 h. The mixture is then diluted in dichloromethane and washed twice with water. The dichloromethane layer is dried over sodium sulfate and evaporated to dryness to give the title compound.

(4-Amino-3-isobutylamino-phenyl)-carbamic acid tert-butyl ester (R from Scheme III with R1=isobutyl, R4=Boc-NH, and R3 and both R5=H): A solution of (3-Isobutylamino-4-nitro-phenyl)-carbamic acid tert-butyl ester (0.51 g; 1 equivalent) in ethanol (15 ml) is combined with a suspension of palladium on carbon (10 weight %) (0.1 g; 2 equivalents) in ethanol (15 ml) while stirring under a nitrogen atmosphere. The nitrogen is then displaced with a positive pressure of hydrogen, and the mixture is stirred for 16 hours at 25 ° C. under hydrogen atmosphere. The mixture is then filtered through celite, and the precipitate is washed with dichloromethane. The filtrate is evaporated to dryness to give the title compound.

6-tert-Butoxycarbonylamino-1-isobutyl-1H-benzimidazole-2-carboxylic acid ethyl ester (S from Scheme III with R1=isobutyl, R4=Boc-NH, and R3 and both R5=H): A 50% solution of ethyl glyoxylate polymer in toluene (0.5 g; 1.5 equivalents) is combined with a solution of (4-amino-3-isobutylamino-phenyl)-carbamic acid tert-butyl ester (0.46 g; 1 equivalent, assuming 100% yield from previous step) in ethanol (3 ml). The mixture is stirred open to the air for ½ hour. A solution of iodine (0.21 g; 0.5 equivalents) in ethanol (1.5 ml) is added, and the mixture is then stirred at 25° C. for 8 hours while open to the air. Sodium thiosulfate (0.41 g; 1 equivalent) in water (2 ml) is added, and the mixture is stirred for ½ hour at 25° C. The mixture is diluted in ethyl acetate and washed with water and brine. The ethyl acetate layer is dried over magnesium sulfate and evaporated to dryness. The product is chromatographed on a silica gel column using 50% ethyl acetate -hexane as the eluant to give the title compound.

6-Amino-1-isobutyl-1H-benzimidazole-2-carboxylic acid ethyl ester (T from Scheme III with R1=isobutyl, R4=Boc-NiT, and R3 and both R5=H): Trifluoroacetic acid (0.5 ml) is added slowly to a solution of 6-tert-butoxycarbonylamino-1-isobutyl-1H-benzoimidazole-2-carboxylic acid ethyl ester (0.12 g) in dichloromethane (4.5 ml). The mixture is stirred at 25° C. for 3 hours. The mixture is then evaporated to dryness, and the residue is dissolved in methanol. The solution in methanol is evaporated to dryness; this is then dissolved in dichloromethane and washed with 2 M aqueous potassium carbonate. The dichloromethane layer is dried over sodium sulfate and evaporated to dryness to give the title compound.

6-Amino-1-isobutyl-1H-benzimidazole-2-carboxylic acid amide (U from Scheme III with R1=isobutyl, R4=Boc-NH, and R3 and both R5=H):A 25% aqueous solution of ammonia (5 ml) is added with stirring to a solution of 6-amino-1-isobutyl-1H-benzoimidazole-2-carboxylic acid ethyl ester (0.02 g) in tetrahydrofuiran (0.5 ml). The mixture is stirred at 25° C. for 72 hours. The mixture is then diluted in ethyl acetate and washed with water. The ethyl acetate fraction is dried over magnesium sulfate and evaporated to dryness to give the title compound.

Scheme IV is another general scheme useful for synthesizing many subject compounds:

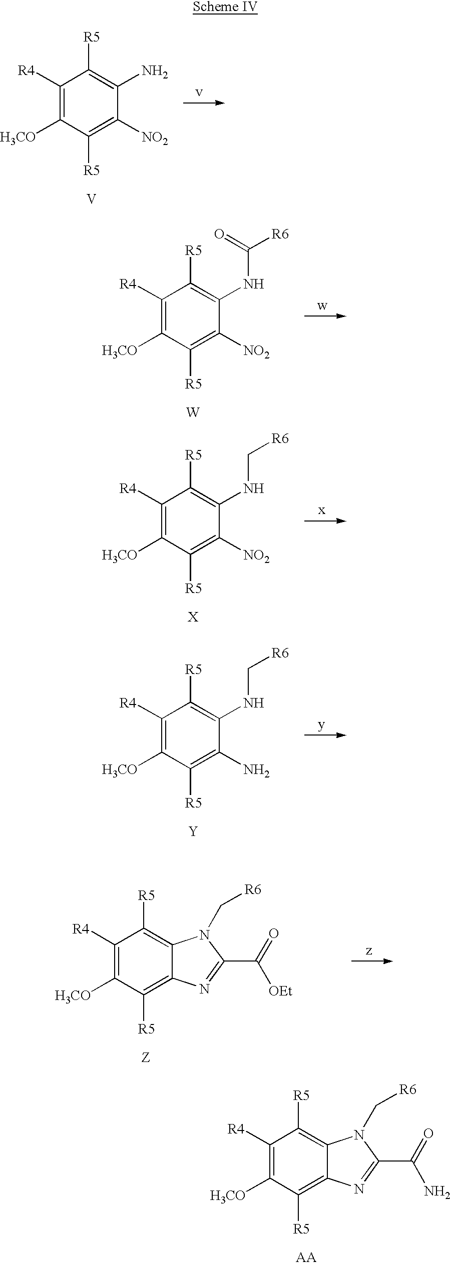

v: R6-COCl, CH₂Cl₂; w: B₂H₆, THF; x: 10% Pd/C, EtOH; y: (CHO—COOEt)ₙ, I₂/EtOH, z: NH₄OH, THF.

N-(4-methoxy-2-nitrophenyl)-isobutyramide (W from Scheme IV with R3=methoxy, R4 and both R5=H, and R6=iPr): 4-methoxy-2-nitroaniline (1.0 g;1 equivalent) and isobutyryl chloride (0.634 g; 1 equivalent) are combined in pyridine (60 mL) and stirred for 4 hours. The mixture is concentrated in vacuo to a residue and dissolved in dichloro methane (DCM) (70 mL), washed twice with 1N HCl (aqueous), saturated Na₂CO₃ (aqueous), water and brine, and dried over MgSO₄. The solvent is removed in vacuo to give 1.35 g of the title compound.

Isobutyl-(4-methoxy-2-nitrophenyl)-amine (X from Scheme IV with R3=methoxy, R4 and both R5=H, and R6=iPr): Isobutyl-(4-methoxy-2-nitrophenyl)-amine (0.1 g ; 1 equivalent) is dissolved in THF (2 mL) and combined with a 1M borane-tetrahydrofuran complex solution in THF (1.25 mL; 3 equivalents) and stirred for 18 hours. Methanol (1 mL) is added slowly, and the solution is heated at 50° C. for 2 hours. The solvent is removed in vacuo and the residue chromatographed (silica gel, 0 to 30% EtOAc/hexanes) to give the title compound.

N¹-isobutyl-4-methoxy-benzene-1,2-diamine (Y from Scheme IV with R3=methoxy, R4 and both R5=H, and R6=iPr): N¹-isobutyl-4-methoxy-benzene-1,2-diamine (0.054 g ; 1 equivalent) is dissolved in ethanol (2 mL) and water (0.1 mL), 10% palladium on carbon (50% wet)(0.011 g) is added. The suspension is stirred under an atmosphere of hydrogen gas for 2 h, and then the flask is purged with nitrogen. The suspension is filtered through a pad of celite, washed thoroughly with DCM, and the combined organic phases dried in vacuo to give the title compound.

1-Isobutyl-5-methoxy-1H-benzoimidazole-2-carboxylic acid ethyl ester (Z from Scheme IV with R3=methoxy, R4 and both R5=H, and R6=iPr): 1-isobutyl-5-methoxy-1H-benzimidazole-2-carboxylic acid ethyl ester (0.034 g; 1 equivalent in ethanol (0.5 mL)) and a 50% solution of ethyl glyoxalate polymer in toluene (0.051 mL; 1.5 equivalent) are combined and stirred for 0.5 hour. A solution of iodine (0.022 g; 0.5 equivalent) in ethanol (0.5 mL) is added, and the reaction is stirred for a further 20 hours. A solution of sodium thiosulphate (0.044 g) in water (0.5 mL) is added, and the mixture stirred for 1 hour, then diluted with EtOAc (20 mL). The mixture is washed with water (×3) and brine, dried with MgSO₄, and the solvents removed in vacuum. The residue is purified by chromatography (silica gel, 5% methanol in DMC) to give the title compound.

1-Isobutyl-5-methoxy-1H-benzoimidazole-2-carboxylic acid amide (AA from Scheme IV with R3=methoxy, and R4 and both R5=H, and R6=iPr): A 25% aqueous solution of ammonia (1 mL) is added with stirring to a solution of 1-isobutyl-5-methoxy-1H-benzoimidazole-2-carboxylic acid ethyl ester (0.0036 g) in THF (1 mL). The mixture is stirred for 50 h, then diluted with EtOAc and washed with water. The organic phase is dried with MgSO₄ and evaporated in vacuo to give the title compound.

Scheme V and VI are two general schemes useful for synthesizing many subject invention compounds:

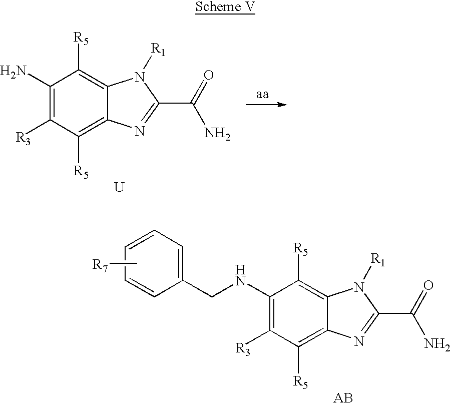

aa: R7PhCHO/AcOH, NaCNBH₃, MeOH

6-[[(4-Methoxy-phenyl)-methyl]amino]-1-isobutyl-1-H-benzimidazole-2-carboxylic acid amide (AB from Scheme V with R1=isobutyl, R3 and both R5=H and R7=methoxy): To a 200 mL round bottom flask 6-amino-1-isobutyl-1H-benzimidazole-carboxylic acid amide (U; 0.1 g) is dissolved in anhydrous MeOH (10 mL). Reagent grade acetic acid (0.1 mL) and 4-methoxy benzaldehyde (0.15 g) is added and the reaction is refluxed until the complete conversion of starting material. At this point the reaction mix is cooled at room temperature and sodiumcyanoborohydrate (0.1 g) is added portion wise. The reaction mixture is stirred at room temperature room temperature for 4 hour. The mixture is then evaporated to dryness and the residue is dissolved in ethyl acetate and washed with 5% aqueous sodium bicarbonate, water, and brine. The organic layer is dried over sodium sulfate and evaporated to dryness. The residue is chromatographed on a silica gel column using 25% ethyl acetate-hexane as eluant to give the title compound.

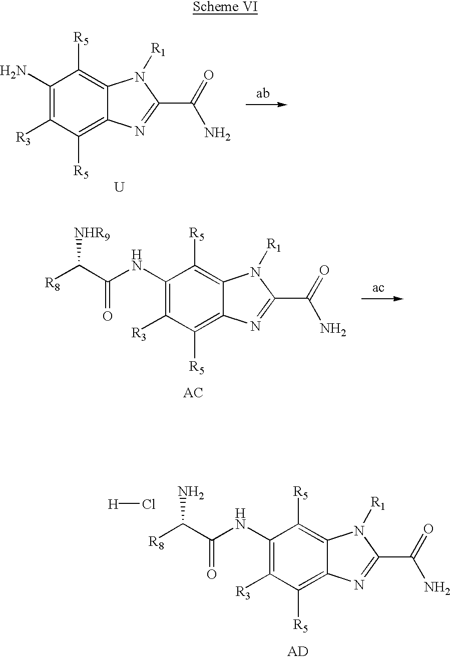

ab: R8-CH(NHR9)COOH, DIEA, EDC, DMAP, HOBT, DCM; ac:1) H₂, 10% Pd/C EtOH; 2) 2N HC

6-{[(2S)-2-(N-Benzyloxycarbonyl)amino-1-oxo-3-phenylpropyl]amino}-1-isobutyl-1H-benzimidazole-2-carboxamide (AC from scheme VI with R1=isobutyl R3 and both R5=H. and R9=Z): N-Benzyloxycarbonyl-L-phenylalanine (64.4 mg) is dissolved in dry dichloromethane (1.5 mL) and N,N-diisopropylethylamine (41.2 μL). 1-(3-dimethylaminopropyl)-3-ethylcarbodiimide (45.4 mg) and 4-dimethylaminopyridine (cat.) are added and the mixture was stirred for 10 minutes at room temperature. 1-Hydroxybenzotriazole (32.0 mg) is then added and stirring was continued for another 5 minutes. Finally, 6-amino-1-isobutyl-1H-benzimidazole-2-carboxamide is added and the resulting mixture is allowed to stir at room temperature for 5 days. The reaction mixture is diluted with dichloromethane and washed with saturated sodium bicarbonate solution (1×), water (1×), and brine (1×). The organic layer is dried and concentrated to a solid. Trituration with methanol gave the title compound as a solid.

6-{[(2S)-2-amino-1-oxo-3-phenylpropyl]amino}-1-isobutyl-1H-benzimidazole-2-carboxamide hydrochloride (AD form scheme VI with R1=isobutyl, R3, both R5 and R9=H). 6- {[(2R)-2-(N-Benzyloxycarbonyl)amino-1-oxo-3-phenylpropyl]amino}-1-isobutyl-1H -benzimidazole-2-carboxamide (84.7 mg,) and 10% Pd/C (17.0 mg) are placed in a flask. In dry ethanol (3.5 mL)the argon line is replaced by a hydrogen balloon. After stirring at room temperature overnight, hydrogen is flushed from the system with argon and the reaction mixture is filtered through a plug of Celite® with 1:1 ethanol/water (100 mL). The filtrate is concentrated to a solid residue. The resulting solid is dissolved in a minimum of methanol and dripped into excess ether to give a slurry. Filtration gave the title compound as a solid.

### The Compositions

The subject invention includes pharmaceutical compositions comprising a safe and effective amount of a 2-carboxamide benzimidazole compound described hereinabove and pharmaceutically-acceptable excipients. The compositions may also optionally include other pharmacologically active compounds, particularly those having activity as thrombolytics (e.g., reteplase, streptokinase or tissue plasminogen activators), anticoagulents (e.g., heparin), beta-blockers (e.g., carvedilol, propanalol), calcium channel blockers (e.g., verapamil, nifedipine), and anti-platelet compounds (e.g., aspirin, abciximab).

Some examples of pharmaceutically-acceptable carriers or components thereof are sugars, such as lactose, glucose, and sucrose; starches, such as cornstarch and potato starch; cellulose and its derivatives, such as sodium carboxymethyl cellulose, ethyl cellulose, cellulose acetate; powdered tragacanth; malt; gelatin; talc; solid lubricants, such as stearic acid, magnesium stearate; or calcium sulfate; vegetable oils, such as peanut oil, cottonseed oil, sesame oil, olive oil, corn oil, and oil of theobroma; polyols such as propylene glycol, glycerin, sorbitol, mannitol, and polyethylene glycol; alginic acid; emulsifiers, such as the Tweens®; wetting agents such as sodium lauryl sulfate; coloring agents; flavoring agents; excipients; tableting agents; stabilizers; antioxidants; preservatives; pyrogen-free water; isotonic saline; and phosphate buffer solutions.

The choice of a pharmaceutically-acceptable carrier to be used in conjunction with a compound is basically determined by the way the compound is to be administered. The compounds and compositions of the present invention may be administered systemically. Routes of administration include topical or transdermal (patch, ointment, cream, powder, etc.); oral; parenteral, including subcutaneous, intramuscular, or intravenous injection; topical; rectal; colonic; intraperitoneal; intraoccular; sublingual; buccal; inhalation; and/or intranasal. The preferred route of administration is parenteral, especially intravenous injection on a daily or as needed basis.

The appropriate amount of the compound to be used may be determined by routine experimentation with animal models. Such models include, but are not limited to the ferret, canine, and non human primate models. Generally, an amount between 0.01 μg/kg to 100 mg/kg of body weight per day is administered dependent on the potency of the compound or compositions used.

Preferred unit dosage forms for injection include sterile solutions of water, physiological saline, or mixtures thereof. Parenteral unit dosage form compositions may be in the form of solutions ready for injection or dry (e.g. lyophilized) compositions which are reconstituted with water or saline solutions prior to injection. The pH of said solutions should be adjusted to about 7.4. Suitable carriers for injection or surgical implants include hydrogels, controlled- or sustained release devises, polylactic acid, and collagen matrices. Other suitable carriers for injection include dextrose, mannitol, lactose, lecithin, albumin, sodium glutamate, and the like.

Compositions of the subject invention are preferably provided in unit dosage form. A unit dosage form composition preferably contains from about 50 mg, more preferably from about 200 mg, also preferably from about 500 mg, preferably to about 2000 mg, more preferably to about 1000 mg, also preferably to about 500 mg, of a 2-carboxamide benzimidazole compound disclosed hereinabove.

The subject compositions may be in a variety of forms suitable (for example) for peroral, topical, or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. The amount of carrier components employed in conjunction with the active compound is sufficient to provide a practical quantity of the material for administration per unit dose of the active compound. Techniques and compositions for making the subject unit dosage forms are described in the following references: Modern Pharmaceutics, vol. 7, chapters 9 & 10, Banker and Rhodes, editors, 1979; Lieberman et al., Pharmaceutical Dosage Forms: Tablets, 1981; and Ansel, Introduction to Pharmaceutical Dosage Forms, 2d edition, 1976.

A preferred dosage form of the subject invention is intended for parenteral administration. Preferred pharmaccutically-acceptable excipients for such compositions include sterile, pyrogen-free water and physiological saline solution. Parenteral unit dosage form compositions may be in the form of solutions ready for injection or dry (e.g., lyophilized) compositions which are reconstituted with water or saline solution prior to injection.

Preferred compositions of the subject invention include those intended for peroral administration, such as tablets, capsules, powders and liquids. Suitable pharmaceutically-acceptable excipients, and anti-platelet compounds (e.g., aspirin, abciximab)nts for such compositions include sugars, starches, cellulose and its derivatives, malt, gelatin, talc, calcium sulfate, magnesium sulfate, vegetable oils, synthetic oils, polyols, algenic acid, phosphate buffers, emulsifiers, alcohols, and water.

### Methods Of Using The Compounds

The 2-carboxamide-benzimidazoles compounds of the subject invention are useful for the treatment of ischemia-reperfusion injury. Although not limited to any specific mechanism, it is believed that the compounds act via modulation of adhesion molecule metabolism. Therefore, the subject compounds are potentially useful for the treatment of ischemia-reperfusion injury including: cardiovascular disease (myocardial ischemia, angina, cardiac arrhythmia, heart failure, hypertension); for treatment to a reduce neurotoxic injury associated with anoxia or ischemia which typically follows stroke, cardiac arrest, or perinatal asphyxia; for treatment to reduce reperfusion injury following organ transplantation; for treatment of frostbite, inflammatory valve disease, psoriasis, asthma, adult respiratory distress syndrome; for treatment of chronic inflammatory lung diseases including emphysema, bronchitis; and for treatment of fibrosis, urticaria, angioedema, vasculitis, migarine, rheumatoid arthritis, gout, and allergy.

It is known that a variety of processes are involved in reperfusion injury, inflammation and related processes. Not being bound by theory, the following mechanism is of interest regarding the subject invention. A key event in the reperfusion injury damage process is the up-regulation, expression, activation of intracellular adhesion molecule-1 (ICAM-1) on endothelial cells. ICAM-1 can then interact with neutrophils resulting in the transmigration of the neutrophils into the tissue with subsequent release of deleterious enzymes and destructive reactive oxygen molecules. Thus, compounds which can interfere with the up-regulation, expression, or activation of ICAM-1 are likely to have a beneficial effect for ischemic reperfusion events. These compounds can be administered via oral, intra-vascular, subcutaneous, intra-musclar, intra-nasal, intra-rectal, intra-occular, sublingual/buccal, inhalation, and topical (patch, ointment, powder, or cream) routes, as long as an effective dose is delivered to the source of the ICAM-1.

Subject invention 2-carboxamide-benzimidazoles, significantly reduce ICAM-1 up-regulation, expression, or activation. In other assays, subject compounds demonstrate activity which correlates with protection to the heart. The following are test methods useful for determining such activities of compounds.

Assay For The Inhibition of Expression of ICAM-1

Tissue: The expression of adhesion of molecules (ICAM-1 in particular) is performed on Human Umbilical Vein Endothelial Cells (HVEC) obtained from Clonetics Corp. (Cat # CC2519), San Diego, Calif.

Endpoint: Concentration of material that inhibits 50% of the expression of ICAM-1 on the surface of HUVECs upregulated with 300 U/ml of TNF-alpha (IC₅₀).

Method: Thaw 1 vial of frozen HUVEC (rapidly at 37° C. for ˜2 min (5×10⁵-1×10⁶ cells in 1 ml medium), then transfer cells to 45 ml pre-warmed growth medium (EGM for HUVEC) in a 225 cm² flask (seeded at 2500-5000 cells/cm²) and place in a humidified 37° C. incubator with 5% CO₂. Change medium after 24-30 h (to remove dead cells and cytopreservatives), and change every 2-3 days thereafter—cells should be confluent after 5-7 days of growth. Trypsinize cells to remove from flask—spin (200 g, 5 min) to pellet cells—resuspend cells in 50 ml medium (want ˜1-2×10⁵ cells/ml) and plate 100 ml cell suspension in 96-well, collagen-coated plates (use ˜1-2×10⁴ cells/well) —grow to confluence (1-2 days). Remove old culture medium (discard) and add 90 ml fresh medium containing TNF-alpha (or other ICAM-1 stimulator at desired concentration) or 90 ml medium alone (for unstimulated control wells). Add 10 ml medium (or PBS or PBS, 1% DMSO) containing compound to be tested ( at 10× desired concentration) or additional 10 ml medium alone (or PBS or PBS, 1% DMSO) for control wells—(final compound concentration=1×; final DMSO concentration=0.1%, if used)—incubate 4 hours at 37° C. Remove medium (discard) and fix cells in 200 ml 80% acetone: 20% H₂O for 20 min at (−20° C.). Remove acetone: H₂O (discard) and allow plates to air dry—and store at (−20° C.) overnight in desiccator. Wash plates with PBS (5×250 ml), then add 200 ml BLOTTO solution to block non-specific binding—incubate 1 hour at room temperature. Remove BLOTTO (discard) and wash plates with PBS (2×250 ml), then add 100 ml ICAM-1 antiserum (in BLOTTO)—incubate 1 hour at room temperature and remove ICAM-1 antiserum (discard) and wash plates with PBS (5×250 ml), then add 100 ml goat anti-mouse-HRP antiserum conjugate (in BLOTTO)—incubate 1 at room temperature. Remove antiserum-HRP conjugate (discard) and wash plates with PBS (5×250 ml), then wash with citrate buffer ( 1×300 μl; discard). Add 100 ml HRP substrate and incubate for 5-20 min at room temperature (time may vary; watch color development), add 50 ml 1N H₂SO₄ to wells to stop reaction. Read plates at 490 nm on plate reader.

Solutions For ICAM-1 ELISA:

(1) 80% acetone:20% H₂O (v:v)—store at (−20° C.).

(2) (1×) Dulbecco's phosphate buffer solution (DPBS), w/o Ca⁺⁺ or Mg⁺⁺, pH 7.5—(Sigma D-5652, 1× powder or Sigma D-1408, 10× liquid, dilute 1:10 before use).

(3) BLOTTO—5% (w:v) non-fat dry milk (Carnation or other) in DPBS.

(4) Mouse anti-human ICAM-1 monoclonal antiserum (Research Diagnostics; catalog #RDI-CBL450-1×; anti CD54-clone 15.2)—stock solution (1 mg/ml)—dilute 1:1000 in BLOTTO just prior to use (1 mg/ml final antiserum concentration).

(5) Goat anti-mouse IgG-horseradish peroxidase conjugate (IgG-HRP, DAKO Corp; catalog # P0447)—stock solution (1 mg/ml)—dilute 1:1000 in BLOTTO just prior to use (1 mg/ml final concentration).

(6) Citrate buffer, pH 5.0—65.3 mM sodium phosphate (dibasic, 12-hydrate; MW=358.4; 23.4 g/l) and 34.7 mM citric acid (anhydrous, free acid; MW=192.1; 6.67 g/l)—check pH (5.0), store at 4° C.

(7) HRP substrate—o-phenylenediamine dihydrochloride (OPD; Sigma, P6912; 5 mg OPD/tablet)—add 1 tablet/10 ml citrate buffer (at room temperature), then add 4 ml 30% H₂O₂ (Sigma, H1009)/10 ml substrate solution just prior to use —final concentration=0.5 mg OPD/ml and 0.012% H₂O₂.

(8) Human tumor necrosis factor alpha (TNF-alpha, Boehringer-Manheim; catalog #1371843)—10 mg/vial (in 1 ml)−10⁸U activity/mg=10⁶ U/10 mg—diluted to 20 ml endothelia cells basal media (EBM) (50000 U/ml; 500 ng/ml)—aliquot 150 ml (7500 U; 75 ng) into eppendorf tubes (×133)—store at 20° C.—for each experiment, add one aliquot to 25 ml EBM−final concentration=300 U/ml or 3 ng/ml TNF-alpha.

Assay For The Inhibition of Human Umbilical Vein Endothelial Cell (HUVEC)/Neutrophil Adhesion

Tissue: Adhesion is performed on Human Umbilical Vein Endothelial Cells (HUVEC) obtained from Clonetics (Cat # CC2519).

Endpoint: Concentration of material that inhibits 20% of the PMN adhesion to HUVECs upregulated with 300 U/ml of TNFalpha (IC₂₀)

Method:

I. Cells:

A. Endothelia Cells:

Human umbilical vein endothelial cells (HUVEC) are purchased as frozen cells in 1 ml aliquots (Clonetics Corporation, San Diego, Calif.). Endothelial Growth Media-Umbilical Vein (EGM-UV), bullet kit additives, trypsinization reagents (trypsin neutralizing solution and HEPES buffer) are also purchased from Clonetics. The flask is placed at 37° C. in a 5% CO₂+95% air, 100% humidity incubator. One vial of liquid N₂ frozen cell is thawed in the 37° C. water bath, the whole vial placed in a T-275 flask with 50 mls of fresh media and placed in the CO₂ incubator. The media is replaced 24-48 hrs later. Confluency should occur within 4-5 days. Media is changed at least once during that period. The monolayer in the flask is detached using the trypsin solution, after the monolayer is washed with Hanks balanced salt solution (HBSS). The trypsinized cells are centrifuged at 200×Gs for 5 minutes and resuspended in approximately 150 mls of media. 100 ul aliquots are placed in each well of 96-well plate that had been previously coated with collagen. The monolayer in the plate should be confluent within 48 hrs.

B. Neutrophil Isolation:

Peripheral blood polymorphonuclear neutrophils (PMNs) are isolated by established methodology (1). Human blood is obtained from the cubital vein by conventional venipucture performed by qualified phlebotomist. The blood is collected in heparinized vacutainers (Vacutainer #6489, green cap, 15 ml draw, VWR). Thirty ml of blood is used for each assay. The heparinized blood is diluted with approximately ½ volume of phosphate buffered saline containing 0.2% glucose (PBS-G). A discontinuous gradient of Histopaque (3 ml of Histopaque-1119 in the bottom and 3 ml of Histopaque-1077 on top) (Sigma Chemical Co., St. Louis, Mo.) is prepared in 6, 15-ml conical centrifuge tubes. The diluted blood is carefully layered on top of the Histopaque-1077. The tubes are centrifuged at 800×G for 30 min at room temperature. After the centrifugation step the PMNs are removed by aspiration from the area between Histopaque-1077/Histopaque-1119 interface and the top of the pelleted red blood cells. The PMNs are collected from all the tubes, further diluted to a total volume of 30 ml and centrifuged at 600×G for 15 min. The supernatant is discarded, and the pellet (containing PMNs and some red blood cells) is treated with 6 ml of cold water for 30 seconds to lyse contaminating RBC. Normal osmolarity is reestablished by adding 3 ml of 2.7% saline. The PMNs are washed an additional 2 times with PBS-G. The viability and number of the PMNs is determined using the trypan blue exclusion test in a hemocytometer counting chamber. Occasionally, a small aliquot of the PMNs suspension is used for a Cytofuge preparation. The Cytofuge slide is stained with Wright's blood stain (Sigma Chemical Co.) and a differential court performed to evaluate the percent of PMNs in the preparation.

II. Upregulation of Endothelia Cells:

The monolayers of endothelial cells in the 96-well plates are upregulated with 300 U/ml of Tumor Necrosis Factor (TNF, Boehinger-Manheim Catalog # 1371-843). The TNF and compound are added to each well 4 hours prior to the addition of the PMNs.PMNs.

III. Fluorescent Labeling of Neutrophils:

After the last wash the neutrophil pellet is resuspended in 5 mls of PBS-G (approximately 1-3×10⁶/ml). 5 (and 6) carboxyfluorescein diacetate succinimidyl ester (CFSE, Molecular Probes, Eugene, Oreg.). A 20 mM stock of the CFSE is prepared by dissolving 25 mg into 2.24 mls (MW557.5) of DMSO. 5 μl of the stock is added to the 5 ml suspension of PMNs for a final concentration of 2 uM. The mixture is incubated in the refrigerator for 20 minutes. At the end of this period the PMNs are washed 4 times with PBS-G. After the final wash the PMNs are resuspended in complete EGM-UV media to the desired concentration (usually each well of a 96-well plate receives 0.6-1.2×10⁵ PMNs).

IV. Addition of Test Compounds:

One hundred μl of media containing the compounds with TNF is used to replace the media in the wells containing the monolayers of endothelial cells 4 hours prior to the addition of the PMNs.

V. Adhesion Assay:

A. Collection of Data:

CFSE-labeled neutrophils (0.7 to 1.5×10⁵) in 10 μl volumes (see III) are added to the HUVEC monolayers. The plates are incubated at 37° C. in a 5% CO₂+95% air, 100% humidity incubator for 30 min. Non-adherent cells are removed by centrifugation according to the following protocol:

(1) A reading is taken in the Cytofluor 2400 after the incubation at 37° C. This reading is considered as 100% of all the cells added.

(2) The wells are filled with warmed (incubator) media to the top of the well with a slight convex meniscus (usually 260 ul (in addition to the 100 ul or so already in the wells)).

(3) The wells are sealed using Adhesive Sealing Films for Micro Plates (Rainin Catalog #96-SP-100).

(4) The lid is replaced on the plate and the size and location of any bubbles recorded on the lids with a marker.

(5) The lids are removed and a folded (4 plies) and cut piece of paper towel placed on top of the Sealing film. The plate lid is then replaced on top of the paper towel and the plate inverted.

(6) The plates are placed on the plate holders of the centrifuge (Sorvall Model RT 6000D) and spun by turning the speed down to around 500 RPMs, then turning the centrifuge on with the timing knob. The speed control is adjusted until the tachometer read 1100 RPM (this is the equivalent of 200 Gs). At the moment that the speed of the 1100 RPM is achieved a separate timer was started. At exactly 2 minutes the timer in the centrifuge is brought to zero to stop the motor. The plates are allowed to come to rest without any braking.

(7) The plates are removed and any empty wells recorded. The lid and the folded paper towel are removed (maintaining the plate upside-down). The Sealing Film is then removed over the biological material disposal bin and the media shaken out. The plate is then blotted on the paper towel and any excess fluid aspirated.

(8) The plate is returned to the upright position and studied under the microscope.

(9) A second reading is taken on the Cytofluor. This second reading is used to determine the percent of PMNs that remained adhered to the monolayer.

(10) The information from the CSV files of the Cytofluor are downloaded and the data processed in a prepared EXCEL spreadsheet where the background (PMNs adherent to non-upregulated endothelium) is subtracted and the percent adhesion inhibition is determined as follows:

$$\begin{matrix}Percent \\ adhesion \\ inhibition\end{matrix} = 100 - \frac{( \begin{matrix}Individual adherent PMNs with \\ background subtracted \times 100\end{matrix} )}{\begin{matrix}Mean adherent PMNs to upregulated \\ endothelium minus background\end{matrix}}$$

(11) The EXCEL spreadsheet calculates a) the percent of PMNs adhering to the monolayer, b) the percent PMNs adhering to the monolayer minus background (PMNs adherent to unstimulated endothelial cells) and c) the percent adhesion inhibition considering the wells receiving TNF alone to the 0% inhibition (negative numbers indicate increase in adhesion).

B. Statistics, Data Handling, and Storage:

Statistics are performed using the double sided t-test with equal variance in EXCEL and the results recorded as the P value.

VI. Additional Information:

A. To Coat Wells With Collagen:

Dissolve 25 mg of acid soluble rat tail collagen 446 mls of water acidified with a few drops of HCl. Sterilize by filtration. Add 50 ul to each well in a 96-well plate (0.28 cm²) for 10 ug/well. Incubate overnight in the 37° C. incubator. Aspirate all the liquid and store in refrigerator until use.

B. To Prepare PBS+0.2% Glucose (Dextrose):

Prepare 2 liters of PBS (Sigma) and add 2 grams of glucose. Sterile filter and save.

Rat Myocardial Infarct/Reperfusion Injury Model

Surgical Preparation of Rats:

Male, Sprague-Dawley rats are anesthetized with urethane, 1.25 g/kg ip. A carotid artery and jugular vein are exteriorized and cannulated with PE-50 tubing for recording blood pressure and to facilitate intravenous administration of dye or drug. A Tracheotomy is performed. The animals are connected to a Harvard Rodent Ventilator (Model 683, Harvard Apparatus, South Natick, Mass.) and ventilated at 1.5 ml/100 g body weight at 50 strokes/min. Needle electrodes are placed for a lead II electrocardiogram. The animals are maintained at 37° C. by means of electric heating pads adjusted to the desired temperature and controlled via a rectal thermistor probe and controller. The heart is carefully isolated by a left thoracotomy at the fifth intercostal space, and the left anterior descending coronary artery (LAD) is located. A ligature of 6-0 silk is placed around the LAD, with the ends threaded through a small length of PE-320 tubing to facilitate rapid occlusion and reperfusion of the artery. The LAD is occluded by clamping the suture and tubing tight against the heart surface using 25 mm Schwarz aneurysm clip. Occlusion lasts for 90 min and is followed by reperfusion for 3.0-4.5 hour. Animals are dosed with drug or vehicle 10 min prior to reperfusion of the affected area of the heart by intravenous delivery via a jugular vein. Sham-operated rats are not subjected to ischemia or reperfusion. At the end of the experiment, the LAD is permanently re-occluded and a 10 mg/ml solution of Evans Blue Stain is administered via the jugular cannula to identify the area affected by ischemia, i.e., the area-at-risk (AAR). The stained heart is rapidly excised and placed into 0.9% saline at 4° C. prior to the determination of creatine phosphokinase activity (CPK).

Determination of Creatine Phosphokinase Activity:

The left ventricular free wall (LVFW) is dissected free from the heart and weighed. The AAR, as defined by the absence of stain, is dissected from the LVFW and also weighed. The AAR is homogenized for 5 sec in 4 ml of 0.25 M sucrose containing 1 mM EDTA and 10 mM mercaptoethanol at 4° C. The homogenate is centrifuged at 3000×g for 30 min at 4° C. The supernatant is decanted for determination of CPK activity and the pellet is stored frozen for the isolation and assay of myeloperoxidase activity. CPK activity is assayed spectrophotometrically with a commercially supplied substrate, CPK Assay Vial® (Sigma Diagnostics), at a wavelength of 340 nm at 24-26° C.

Determination of Myeloperoxidase Activity:

Myeloperoxidase (MPO) is isolated from the frozen pellet after the preparation of CPK. The pellet is suspended in 50 mM phosphate buffer, pH 6, containing 0.5% hexadecyltrimethylammonium bromide (HTAB) to a concentration of approximately 10% sonicated for 10 sec and frozen on dry ice. Three freeze-thaw cycles are done with 10 sec of sonication between cycles. The samples are chilled on ice for 30 min followed by centrifugation at 12,500×g for 15 min at 4° C. An aliquot of the supernatant is assayed spectrophotometrically for MPO activity in 50 mM sodium phosphate buffer, pH 6, containing 0.167 mg/ml o-dianisidine dihydrochloride and 0.113 ml hydrogen peroxide per 100 ml buffer at a wavelength of 460 nm at 24-26° C.

Calculations and Statistical Analysis:

The results are reported as the mean ±SEM. CPK and MPO activity are expressed as units/g tissue, where 1 unit of CPK activity is defined as the quantity of CPK utilizing 1 μmol phosphoceatine per minute and 1 unit of MPO activity is that quantity that degrades 1 μmol peroxide per minute. The AAR is quantified as a percentage of the LVFW based on weight. Mean arterial blood pressure (MABP) is calculated as one-third the difference between systolic and diastolic blood pressure added to diastolic blood pressure. Data are analyzed for statistical significance of treatment effects at the 95% confidence level by a pooled t-test or by one-way analysis of variance.

The subject invention involves methods of treating or preventing any of the diseases and disorders provided hereinabove, especially reperfusion injury, by administering a safe and effective amount of a 2-carboxamide benzimidazole compound disclosed above. Such methods of treatment can involve administering a unit dosage form of such compounds parenterally, perorally, or topically. Parenteral administration includes intravenous, intramuscular, subcutaneous, intraperitoneal, or other injection of the dosage form. Peroral administration involves ingestion of the dosage form and absorption of the active from the gastrointestinal tract. Topical administration involves contacting the dosage form with the surface of the skin or mucosal tissues, including, but not limited to, those of the alimentaryl canal and the respiratory system.

For parenteral administration, the amount of 2-carboxamide benzimidazole compound typically administered is preferably from about 2 mg/kg, more preferably from about 5 mg/kg, preferably to about 2 mg/kg, more preferably to about 10 mg/kg. The frequency of such administration is typically once or twice daily. A treatment regimen typically is a single dose, or lasts from about 1 day, preferably from about 5 days, to about 30 days, preferably to about 15 days.

For peroral administration, the amount of 2-carboxamide benzimidazole compound typically administered is preferably from about 5 mg/kg, more preferably from about 10 mg/kg, preferably to about 25 mg/kg, more preferably to about 15 mg/kg. The frequency of such administration is typically from once to about 4 times daily. A treatment regimen typically lasts from about 1 day, preferably from about 5 days, to about 30 days, preferably to about 15 days.

### COMPOSITION AND METHOD EXAMPLES

The following non-limiting examples illustrate the subject invention. The following composition and method examples do not limit the invention, but provide guidance to the skilled artisan to prepare and use the compounds, compositions, and methods of the invention. In each case other compounds within the invention may be substituted for the example compound shown below with similar results.

### Example A

Pharmaceutical compositions in the form of an intravenous solution are prepared by conventional methods, such as mixing the following:

| Ingredient | Quantity (mls) |
|---|---|
| Compound of Example 1¹ | 1 mg. |
| Sterile water | 10 ml |
| HCL and/or NaOH | pH 7.2-7.5 |

When 1 ml of the above composition is administered intravenously, either immediately before or immediately after a tissue damage event (aneurysm repair, coronary bypass, transplant surgery, traumatic hemorrhage, organ ischemia due to hypoperfusion, sepsis, etc.), tissue damage is avoided or reduced.

### Example B

Pharmaceutical compositions in liquid form are prepared by conventional methods, formulated as follows:

| Ingredient | Quantity |
|---|---|
| Compound of Example 1² | 1 mg |
| Phosphate buffered physiological saline | 10 ml |
| Methyl Paraben | 0.05 ml |

When 1.0 ml of the above composition is administered subcutaneously, either immediately before or immediately after a tissue damage event (aneurysm repair, coronary bypass, transplant surgery, traumatic hemorrhage, organ ischemia due to hypoperfusion, sepsis, etc.), tissue damage is avoided or reduced.

While particular embodiments of the subject invention have been described, it would be obvious to those skilled in the art that various changes and modifications to the compositions disclosed herein can be made without departing from the spirit and scope of the invention. It is intended to cover, in the appended claims, all such modifications that are within the scope of this invention.

¹Compound of Example 1 can be substituted with any of Examples 2-14.

²Compound of Example 1 can be substituted with any of Examples 2-14.

What is claimed is:

1. A compound having the structure: 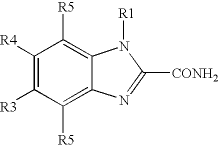
wherein:
(a) R1 is selected from the group consisting of alkyl, aryl, alkoxy, and aryloxy, the alkyl and aryl portions of such R1 having from 1 to 14 carbon atoms;
(b) R3 and R4 are independently selected from the group consisting of hydrogen, halo, alkyl, alkoxy, alkyl-aryloxy, alkylthio, amino, and mono- or dialkylaminio, the alkyl portions of such R3 and R4 having from 1 to 8 carbon atoms; except that R3 and R4 are not both hydrogen;
(c) each R5 is independently selected from the group consisting of hydrogen, halo, cyano, alkyl, hydroxy, alkoxy, thio, alkylthio, amino, and mono- or dialkylamino, the alkyl portions of such R5 having from 1 to 8 carbon atoms; and
an optical isomer, diastereomer, or enantiomer or mixture thereof; a pharmaceutically-acceptable salt, hydrate, or biohydrolyzable ester, amide or imide thereof.

2. The compound of claim 1 wherein R1 is alkyl or aryl having from 2 to 8 carbon atoms; preferably R1 is alkyl having from 3 to 8 carbon atoms, the alkyl being unsubstituted or substituted with substituents selected from the group consisting of halo, hydroxy, C₁-C₃ alkoxy, thio, C₁-C₃ alkylthio, amino, C₁-C₃ mono- or dialkylamino, phenyl, and heterocycle having 5 or 6 ring atoms, more preferably R1 is saturated or unsaturated with one double bond, linear or branched or cyclic alkyl having from 3 to 6 carbon atoms.

3. The compound of claim 2 wherein R3 and R4 are independently selected from the group consisting of hydrogen alkyl, alkoxy, alkylthio, amino, and mono- or dialkylamino, the alkyl portions of such R4 having from 1 to about 6 carbon atoms;

4. The compound of claim 3 wherein each R5 is independently selected from the group consisting of hydrogen, halo, alkyl, alkoxy, alkylthio, and mono- or dialkylamino, the alkyl portions of such R5 having from 1 to 6 carbon atoms.

5. The compound of claim 4 wherein the alkyl portions of the R3, R4 and R5 moieties independently have from 1 to 3 carbon atoms, unsubstituted or substituted with halo, hydroxy, C₁-C₃ alkoxy, thio, C₁-C₃ alkylthio, amino and C₁-C₃ mono- or dialkylamino, preferably the alkyl portions of R₃, R₄, R₅ are unsubstituted.

6. The compound of claim 5 wherein R1 is phenyl or benzyl, unsubstituted or substituted with substituents selected from the group consisting of halo, C₁-C₃ alkyl, hydroxy, C₁-C₃ alkoxy, amino, and C₁-C₃ mono- or dialkylamino, preferably R1 is saturated and unsubstituted, and preferably R3 methoxy or ethoxy.

7. The compound of claim 6 wherein R4 is alkoxy, preferably methoxy or ethoxy, more preferably methoxy and R3 and both R5 are hydrogen.

8. The compound of claim 7 wherein R3 is alkoxy, and R4 and both R5 are hydrogen.

9. A pharmaceutical composition comprising:
(a) a safe and effective amount of a compound of any of claim 1 or 6; and
(b) pharmaceutically-acceptable excipients.

10. A method of preventing or treating an ischemia-reperfusion injury comprising administering to a human or lower animal in need thereof, a safe and effective amount of a compound of any of claim 1 or 6.
